(19) United States
(12) Patent Application Publication
(10) Pub. No.: US 2021/0077647 A1
(43) Pub. Date: Mar. 18, 2021

(54) Air freshener promotional product

(71) Applicant: Cora Keith, Poughkeepsie, NY (US)

(72) Inventor: Cora Keith, Poughkeepsie, NY (US)

(21) Appl. No.: 16/602,326

(22) Filed: Sep. 16, 2019

(51) Int. Cl. A61L 9/12; G09F 23/00

(52) U.S. Cl. CPC A61L 9/12; A61L 2209/13; G09F 23/00

(57) ABSTRACT

An air freshener promotional product comprising a hanging handbag air freshener being a miniature replica of a handbag having a height/ratio of from 2 to 3 inches, being a representation of a type of handbag has a promotional strap upon which graphics, can be displayed said miniature replica of a can be designed and embellished to resemble stylish handbags wherein handbag replica is hung from a rearview mirror of a car or a hang point and is used as an ornamental air freshener.

### FIELD

The following application relates to a promotional product, including a miniature replica of a handbag suspended from a chain or cord made to be suspended from a rearview mirror of a car and being a representation of a type of handbag including a cross body bag, a messenger bag, a tote bag, a bucket bag, a hobo bag. The handbag having interchangeable, straps including one strap upon which logos, slogans, messages, company names, personal messaged can be displayed having a housing capable of receiving a scent dispensing unit, wherein, the hanging device is used as an air freshener.

### BACKROUND

It is well known that promotional products have been used as a cost effective marketing method for companies and businesses. These items are used as giveaways and are a means of reaching out to potential customers. Customers like to receive promotional products that has an advertising message. A large percent of customers who receive promotional products do business with the advertisers. The customers usually keep the promotional product for years after receiving the product and are able to recall the companies name for months after receiving the item, and more referrals are gained form customers who are satisfied with the gift items. This makes the use of promotional products for marketing and branding recognition very significant in a competitive market. These products have logos, messages, slogans printed on them. The items are usually given away at tradeshows and different events. And are usually items of daily use. These, gifts may be calendars, pens drink wear, lip balms and air fresheners, and the like. Marketers employ a creative strategy when choosing these items that are not only useful but unique for a wider range of impact. The gift remains in a customer's office home or car for years, reminding them of a business or company and their services. Businesses and companies are choosing promotional products that are unique and attractive and items that are more company and event specific when it comes to providing services to a specific target market. It is also well known that marketers have to compete to gain attention of their target market, in order to make an impact. When a company target market is directed toward the female population the promotional products they choose need to invoke emotional responses for the recipients. Customers, also want to receive promotional products that are relevant to their interest. It is well known that car air fresheners have been used as a promotional product for a very long time. The most commonly type used are paperboard air fresheners that can be seen hanging from rearview mirrors of automobiles. These type of air fresheners come in a variety of shapes having a vast range of scents, and designs, some more appealing to one particular group of the population than others. These air fresheners are inexpensive to purchase and continue to be sold widely in gas stations, carwashes, and novelty stores. However, as a promotional product wherein a company target is directed toward female consumers, paperboard air fresheners are less desirable, due to the fact that women today are seeing their vehicles as an expression of themselves and their personalities. They are in the market for air fresheners that are more novel and personal and desire alternatives to their prior art paper example that have been given out by companies for years and are so wide spread that they don't have a significant meaning or personal emotional connection especially with the specific female population Women are choosing more novel, unique attractive, aesthetically pleasing ornamental air fresheners to accessorize their vehicles. The present application is a miniature replica of a handbag an accessory commonly used and, recognized, by all women. It is known—that women are the major purchasers of the handbag accessory for reasons of keeping up with the times, making a fashion statement, for personal style, elegance, or social status, for whatever the reason women purchase handbags there is a matter of importance for this particular accessory. It has been advantageous to develop an air freshener promotional product that combines form and function and is aesthetically pleasing and, is emotionally relevant to the interest of women. Provision of such a construction is a stated objective of the present invention. The present invention provides, economical benefits to companies and businesses that provide services to women. The representation of a miniature replication of a handbag, a fashion accessory used as a promotional product comprising interchangeable straps that can be attached to the handbag wherein at least one strap providing a smooth surface upon which Logos, messages, slogans, company name and personal messages can be displayed, other straps made of metal components that can be attached to the handbag for advertising purpose or for decorative purpose. The miniature handbag is a hanging ornamental item that is capable of housing an air freshener dispensing unit. A promotional product with the aforementioned features is a fun and novel and entertaining way to advertise a company or business. The item can be used to promote handbag companies, air freshener companies. Spas, Private organizations for women etc. Businesses who provide services to women will appreciate the ability to add logos, messages slogans, to a functional and useful product that economically can be a low budget or high end product. There are various promotional products that do not provide the novelty of the present invention.

### SUMMARY

An objective of the present invention is the provision of a promotional product comprising, a miniature replica of a handbag wherein a handbag strap upon which logos, company names slogans and personal messages can be displayed

Another objective is to provide an entertaining and fun promotional product that can be hung from a rearview mirror having a decorative appearance that is appealing to women.

Still another objective is to provide a promotional product that can be used by companies and businesses who provide services to women.

Features of the present invention is the provision of a promotional product including a miniature replica of a handbag, that is used as an air freshener comprising at least one strap for advertising a company name ,slogan, personal messages.

### BREIF DISCRIPTION OF DRAWING

Above-mentioned features and objectives of the present invention will become more apparent by the reference to the following description taken in conjunction with the accompanying drawings. In which:

FIG. 1 is a front view of the promotional product in accordance with the principles of the present invention.

FIG. 2 is a back view of the promotional product in accordance with the principles of the present invention.

FIG. 3 is a view of the handbag promotional strap in accordance with the principles of the present invention.

FIG. 4 is a front view of the handbag with promotional strap attached to the handbag in accordance with the principles of the present invention.

FIG. 5 is an inside view of the back of the handbag containing a scent dispensing unit according to the principles of the present invention.

FIG. 6 is a view of metal strap for attachment to the handbag in accordance with the principles of the present invention.

FIG. 7 is a view of metal strap for attachment to the handbag in accordance with the present invention. FIG. 8 is a view of metal strap for attachment to the handbag in accordance with the principles of the present invention.

### DETAILED DISCRIPTION OF DRAWINGS

Before the present invention is described in further detail, it is to be understood that the intervention is not limited to the particular embodiments described herein, it is to be understood that the terminology used herein is for the purpose of describing particular embodiment wily by the, appended claims.

It is to be understood that although methods similar or equivalent to those described herein can also be used in the practice of testing of the present invention, a limited number of exemplary methods are described herein.

It should be understood that the terms and the like, herein do not denote any order ranking or importance, but rather are used to distinguish one element from another. Further the term an, and a herein do not denote a limitation of Quantity, but rather denote the presence of at least one of the referred item.

The description herein is made only by way of example and not as a limitation to the scope of the invention, various modifications and changes could be made there unto without departing from the scope of the present invention.

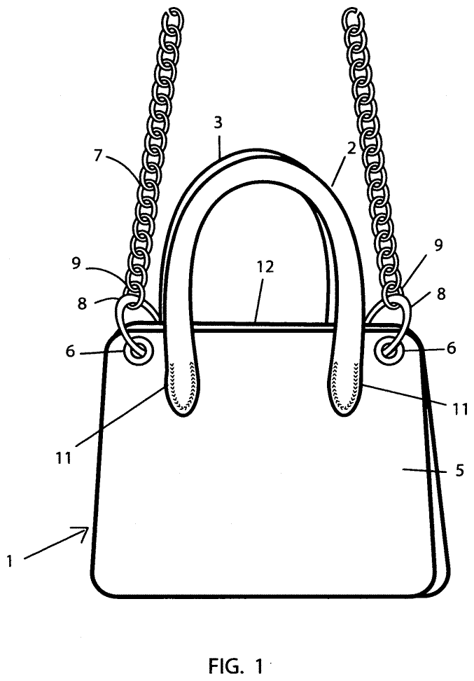

Referring to FIG. 1 the promotional product in accordance with the principles of the present invention includes a miniature replica of a handbag 1 capable of being hung from a rearview mirror of a car. The miniature replica of a handbag 1 can also be used in other settings for decoration, and air freshener use in an office, home etc. The miniature handbag 1 is made in small proportions as not to block the View of a driver of a vehicle. The handbag 1 can be constructed of Faux, leather, cloth, fabric and other similar materials of which handbags are made. The handbag can be a variety of shapes and colors and a variety fabrics with plain or printed patterns can be used to make the handbag. The outside of the Handbags can be embellished to resemble real handbags, using handbag hardware, made of metal plastic, such as snaps, buckles, Rheine stones, snaps, etc. To the benefit of an advertiser fabrics containing logos can also be used to make the handbag 1. The Handbag 1 is formed by joining at least two sides together by means of stitching. The use of adhesives or other means of joining the handbag together to form a handbag shape can include but not limited to the use of stapling or gluing etc. there is further shown in FIG. 1 the illustrated handbag 1 has at least two handles shown as 2 and 3 of FIG. 1. The handles 2 and 3 are attached to the handbag 1 by means, of fastening to the front and rear surface on the handbag 1. For illustration purposes the handle 2 is shown attached to the front surface 5 of FIG. 1 handle 3 is attached to the rear surface of the handbag attachment not shown herein however handle 3 is attached to the rear surface of handbag 1 in the same manner as handle 2 is attached to the front surface 5 of the handbag 1. As best can be seen in FIG. 1 handle 2 is fastened by the use of stitching 11 as shown in FIG. 1 to the front surface 5 of the of the handbag 1. Other means of attaching the handles 2 and 3 can include but not limited to the of use fasteners such as rivets screws snaps, clamps etc. These types of fasteners can be made of metal, plastic, resin materials. The use of adhesives may also be used to secure handles to the handbag 1. Handles 2 and 3 can be made of fabrics, textiles, and the like and can include metal materials, plastic materials and resin pieces to construct handles. Placement of the handles 2 and 3 are not limited to location illustrated, the top portion 12 of the handbag can Also, be used for attaching handles to the handbag 1 according to other designs of the handbag. The miniature handbag 1 of FIG. 1 there is shown a chain 7 attached to the handbag 1 for suspending the handbag in a vertical position, an apparatus used for suspending the handbag is not limited to a chain 7 having links that can be Connected, to the handbag 1. Other cords made of thread like elements wherein the cord provides an attachment means at the ends of the cord such as fold over clamps, clasps, hooks Jump rings and other fastening components that allow for a connection to a fastener engaged with a fastener on the handbag surface for suspending the handbag 1 in a vertical position can be used. FIG. 1. There is shown herein the placement of a grommet 6 attached to the surface of the handbag 1. Grommet 6 is engaged with jump ring fastener 8 and engaged with link of chain 9 for suspending the handbag from a chain 7, Other means of connecting the chain 7 to the hand bag 1 may be used such as attaching a type of fastening means to the surface of the handbag 1 such as applying clamps, pins, hooks, snaps, clasps and other fastening, components to the surface 5 of the handbag 1 to connect with a chain7 or hanging cord.

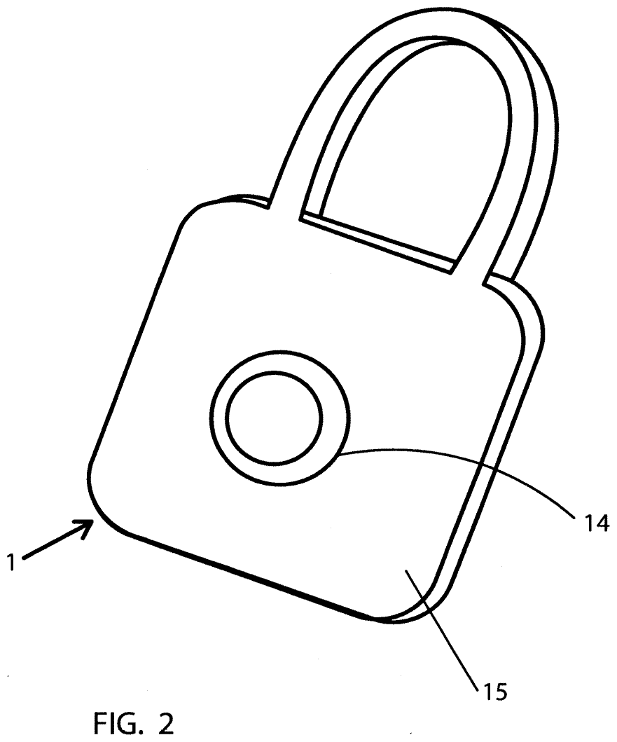

Referring to FIG. 2 there is shown a view of the rear surface 15 of the handbag 1, further showing an aperture 14 that allows scent release from a scent dispensing unit when placed in the handbag 1. The handbag 1 includes at least one aperture 14 in the handbag for allowing release of scent from the housing when a scent dispensing unit is placed inside.

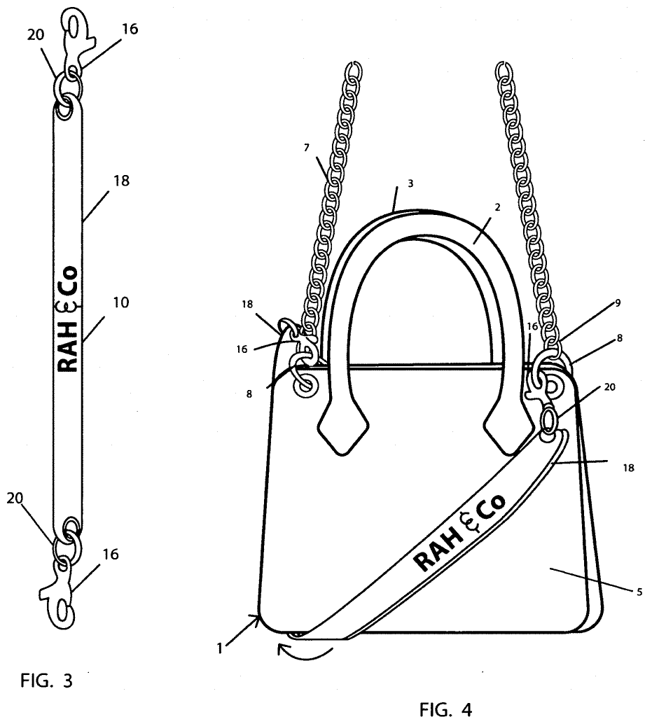

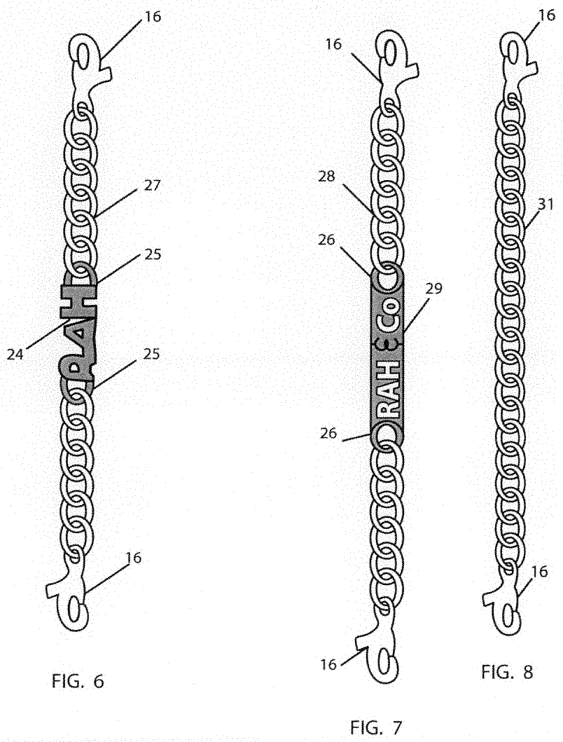

FIG. 3 is a front view of the handbag promotional strap 18 upon which logos, messages, slogan, a company name, personal messages can be displayed. The illustration shows The handbag strap 18 provides strap with a smooth surface 10 to allow for messages to be displayed on the front surface of the strap 18 FIG. 3 further shows the two ends of the strap, 18 are connected to a clasp fastener 16 for engaging a fastener in connection to the handbag 1 as shown 8 of FIG. 3. The materials for making the strap 18 can include but not limited to faux leather, leather, rubberized materials, woven fabrics, with smooth surfaces that allow for printing of message slogans, indicia, straps can also be made of chain configurations and metals wherein custom metal plates can be engraved with messages, slogans, logos, and also sheet metal can be laser cut to form words, slogans or a message for advertising a business or service These straps not shown herein but can be seen in FIG. 6 and FIG. 7 and FIG. 8.

FIG. 4 the Handbag strap 18 is shown to be connected to the handbag 1 by clasp fastenerl6 at one end of strap 18 wherein clasp connector 16 is engaged with jump ring

Fastener, 8 near the top corners of the handbag 1. The opposite end of the strap 18 is folded under the opposite end of the handbag 1 connecting to the jump ring 8 from the back side of the handbag 1 with clasp connector 16 at the other end of the strap. The handbag strap 18 is not limited to being connected by means illustrated herein however to the use of clamps pins, hooks, snaps, clasps and other connecting components placed at the ends of the strap for connecting to the handbag 1.

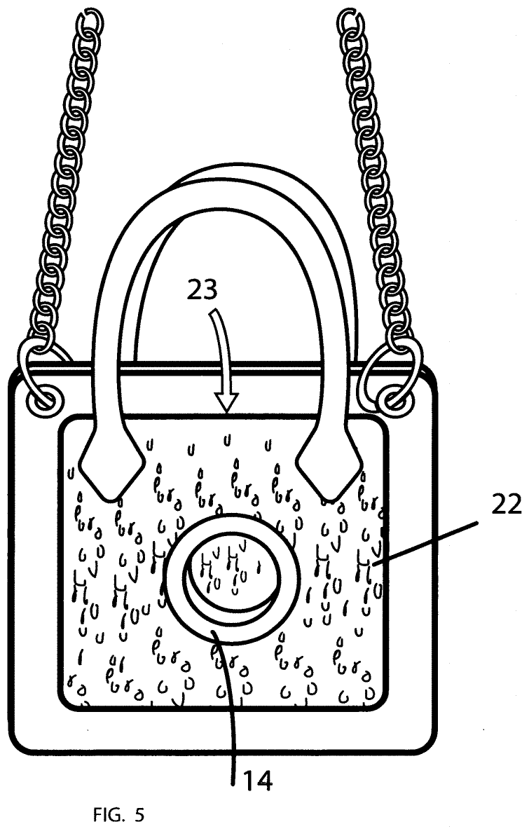

FIG. 5 as best can be seen is an inside view of the back of the handbag 1 wherein the handbag 1 contains a scent dispensing unit 22. The handbag 1 can accommodate a variety of aromatic scent dispensing units, such as a sachet, scent pads, gel type air fresheners, and liquid, and wax type air fresheners units in leak proof containers. The scent dispensing unit 22 is inserted into an opening in the handbag 1 as illustrated herein. The scent dispensing unit 22 is inserted into a top opening 23 of the handbag 1 other openings can be used in the bag for insertion of the scent dispensing unit 22. Openings can be provided at the back surface of the handbag 1. FIG. 5 further shows an aperture 14 in the back of the handbag for allowing scent to be released from a scent dispensing unit placed in the handbag, at least one aperture is provided for scent to be released from the scent dispensing unit 22. Apertures are not limited to be placed only in the rear of the handbag, the sides can also be used as a location for apertures depending on the design of the handbag.

FIG. 6 there is shown a strap 27 made of metal components that can be added to the handbag 1. FIG. 6 is illustrating a chain strap 27 that can be attached to the handbag 1 made of metal materials that have been fabricated to display a company name. A chain strap 27 is illustrated herein with a metal plate 24 that has been fabricated by cutting a message into the metal by a process known as laser cutting and is attached to the chain 27 by metal connecting components 25 at each end of the plate. A connection is provided at the ends of the chain 27

as can be seen by use of clasps 16 for attachment to a handbag 1 attachment for chain 27 used as a strap is not shown herein in FIG. 7 however chain 27 used for strap is connected to the handbag in the same manner as shown in FIG. 3 for strap attachment to the handbag 1.

FIG. 7 there is shown a strap 28 made of metal components that can be attached to the handbag 1 further illustrated in FIG. 7 is a metal plate 29 with an engraved company name. The metal plate 29 is attached to a chain 28 by connecting Components 26 at each end of the plate 29 and is engaged to the chain 28. FIG. 7 further show clasp 16 connectors are provided at each end of the chain 28 for connection to the handbag 1. Attachment of chain 28 used as a strap is not shown attached to the handbag 1 herein however is connected to the handbag 1. In the same manner as shown in FIG. 3 for strap attachment to the handbag.

FIG. 8 as best can be seen is a strap 31 made of metal components. The strap can be used for decorative purpose. A connection is provided at each end of the strap3l as can be seen by 16, however attachment for the strap is not shown herein. The attachment of the strap 31 to the handbag 1 is connected to the handbag in the same manner as shown in FIG. 3 for strap attachment to the handbag 1.

1. A promotional product comprising a women's accessory. The promotional product a miniature replica of a handbag being one of a tote bag, bucket bag, cross body bag, messenger bag, hobo bag, shoulder bag characteristics for advertising a business or company wherein said handbag has an attachment provided for a strap upon which logos, messages , slogans, and company names can be displayed and the said handbag having at least one aperture in the handbag for scent release from a scent dispensing unit, place inside the said handbag for the use as an air freshener.

2. A promotional product according to claim 1, wherein
Said miniature replica of a handbag includes at least one strap upon which logos, messages, slogans, company name, personalized messages can be displayed.

3. A promotional product of claim 2, wherein
Said strap of said miniature replica of a handbag includes at least one smooth surface upon which logos, slogans, messages, company name, personalized messages can be displayed.

4. A promotional product of claim 1, wherein
Said miniature replica of a handbag is used as a hanging ornamental air freshener.

5. A promotional product according to claim 4 wherein
Said miniature replica of a handbag provides at least one aperture in said miniature replica of a handbag housing to allow for scent release from aromatic articles placed inside the handbag.

6. A promotional product according to claim 1, wherein Said miniature replica of a handbag is suspended from a chain in an upright position.

7. A promotional product of claim 1, wherein Said miniature replica of a handbag is suspended by a cord made of thread like elements in an upright position.

8. A promotional product of claim 1, wherein
Said miniature replica of a handbag is a representation of a hobo bag, bucket bag, cross body bag, messenger bag, or shoulder bag.

9. A promotional product of claim 1, wherein
Said miniature replica of a handbag can include at least one strap made of metal components.
